(19) United States
(12) Patent Application Publication
(10) Pub. No.: US 2007/0010760 A1
(43) Pub. Date: Jan. 11, 2007

(54) Binding and removal of contaminants and other chemical agents through novel enhanced carbon-based filtration methods, processes and products

(76) Inventor: Paul Rosenfeld, Venice, CA (US)

Correspondence Address: GREENBERG TRAURIG LLP, 2450 COLORADO AVENUE, SUITE 400E, SANTA MONICA, CA, 90404, US

(21) Appl. No.: 11/323,010

(22) Filed: Dec. 30, 2005

Related U.S. Application Data
(63) Continuation of application No. 11/153,818, filed on Jun. 15, 2005.

(51) Int. Cl. A61M 1/06

(52) U.S. Cl. 600/573; 604/74

(57) ABSTRACT

With a carbon-based filtration system, binding and removal of contaminants and other chemicals is accomplished whereby breast milk may be substantially improved in terms of noxious chemicals and toxic elements reducing the likelihood of transferring body burdens in infants.

### CROSS-REFERENCE TO RELATED APPLICATIONS

This application is a continuation of, and claims the prior filing date of U.S. patent application Ser. No. 11/153,818 filed Jun. 15, 2003, herein incorporated expressly by reference in its entirety, as if set forth herein.

### REFERENCES

Abadin, H G., B F Hibbs and H R Pohl. “Breast-feeding exposure of infants to cadmium, lead, and mercury: a public health viewpoint.” Toxicol Ind Health. 1997.
Dorea, Jose. “Mercury and lead during breast-feeding.” British Journal of Nutrition. 2004. 92:1.
Gundacker C, Pietshnig B, Wittmann K, Lischka A, Salzar H, Hohenauer L, and Schuster E. “Lead and Mercury in Breast Milk”. Pediatrics. 2002. 110:5.
Ip, Henrietta Man Hing. “Breast Milk Contaminants in Hong Kong.” The Bulletin of the Hong Kong Medical Association. 1983. 36.
Jaga, Kushik and Chandrabhan Dharmani. “Global Surveillance of DDT and DDE levels in human tissues.” International Journal of Occupational Medicine and Environmental Health. 2003. 16(1): 7-20.
Kalantzi O, Martin F L, Thomas G O, Alcock R E, Tang H R, Drury S C, Carmichael P L, Nicholson J K, and Jones K C. “Different Levels of Polybrominated Diphenyl Ethers (PBDEs) and Chlorinated Compounds in Breast Milk from Two U.K. Regions”. Environmental Health Perspectives. 2004. 112:10.
LaKind, Judy, Cheston M. Berlin, and Daniel Naiman. “Infant Exposure to Chemicals in Breast Milk in the United States: What We Need to Learn From a Breast Milk Monitoring Program.” Environmental Health Perspectives. 2001. 109:1.
Leeuwen, Rolaf. “WHO Exposure Study on the Levels of PCBs, PCDDs, and PCDFs in human milk.”
Oskarsson, Agneta. “Exposure to Toxic Elements via Breast Milk”. Analyst. March 1995, Vol. 120.
Paccagnella, B., M. Riolfatti. “Total mercury levels in human milk from Italian mothers having not been particularly exposed to methyl-mercury.” Ann Ig. 1989
Plockinger, B., C. Dadak, V. Meisinger. “Lead, mercury and cadmium in newborn infants and their mothers.”
Roots, Ott. “Persistent Organic Pollutants Levels in Human Milk and Food.”
Ryan J. “Polybrominated diphenyl ethers (PBDEs) in human milk; occurrence worldwide”
Smith, Daniel. “Worldwide trends in DDT levels in human breast milk”. International Journal of Epidemiology. 1999. 28:179-188.
“Body Burden The Pollution in Newborns” by Environmental Working Group Jul. 14, 2005
“DDT Health and Safety Update”. Conserve 0 Gram. 2000. 2:14.
“Healthy Milk, Healthy Baby” Natural Resources Defense Council.
“Human Breast Milk is Contaminated” Environmental Research Foundation August 1990
“Lead” Environmental Health Center
“Levels of DDT metabolites in maternal milk and their determinant factors.” Archives of Environmental Health, March 1999
“Mercury Fact Sheet”. Department of Health Services.
“Mercury in Fish”. Department of Heath Services.

### BACKGROUND OF THE DISCLOSURE

A study conducted by the National Academy of Sciences suggests that 28 percent of childhood disabilities can be attributed to environmental factors. In the womb, babies depend solely on nutrients and fluids from their mother. Even after birth, they depend on essential nutrients from breast-feeding, but these nutrients and fluids have built up with dangerous chemicals over generations of environmental pollution. In many cases these contaminants cause detrimental health effects in children, according to accepted scientific literature (see references). Recently there has been an increase in numerous childhood diseases. Autism has increased by a factor of 10, male birth defects and childhood asthma by a factor of 2, acute lymphocytic leukemia by 62%, and childhood brain cancer by 40%. Pollutants that have built up in the human body may not be the sole cause of this increase, but they may have contributed to this growing problem.

In an article published by The Environmental Working Group, an average of 200 industrial chemicals and pollutants were found in the umbilical cord blood of babies born in the US. In total, 287 different chemicals were identified including 180 that cause cancer in humans or animals, 217 that are toxic to the brain and nervous system, and 208 that cause birth defects. Chemicals found include pesticides, consumer product ingredients, and wastes from burning coal, gasoline, and garbage. Even after birth, pollutants continue to contaminate infants at an alarming rate through breast milk. Maternal milk is the main path through which toxic substances are eliminated from the body. As a result, concentrations are extremely high and even surpass the FDA's standards for cow's milk set for adults (FIG. 11).

Regardless of infants' decreased vulnerability after birth, these chemicals still cause greater harm to infants than adults. The risk associated with children's chemical exposures is greater pound for pound. The immature and porous blood-brain barrier allows chemical exposure to the brain. Children have lower levels of some chemical binding proteins which correlate with more chemicals reaching target organs. Baby's organs are rapidly developing and are more vulnerable to damage. In addition, systems that detoxify are not fully developed in infants.

However, the advantages of breastfeeding still outweigh the health risks posed by these contaminants. Breast milk contains essential nutrients that are specifically designed for developing babies and provide essential building blocks for the immune system and growth. A study reported breast-fed infants achieve motor milestones at an earlier age than formula-fed infants. Additional studies find uses of other types of milk as a replacement for breast milk result in a reduced verbal intelligence quotient. Protective factors in breast milk can even counteract some negative effects of contaminants. Nevertheless, pollutants in breast milk negatively affect the milk's nutritional and protective value.

Mothers can limit the contaminants in their milk by removing certain foods from their diet which may contain high concentrations of these contaminants. However, many of these chemicals are already stored in the body and while we can lessen the amount of contaminants we add through our dietary intake, most of the contaminants will still end up in maternal milk.

Two of the most toxic chemicals found in maternal milk are polybrominated diphenyl ethers (PDBEs) and polychlorinated biphenyls (PCBs). Some of the sources of PBDEs include flame retardants in furniture foams, computers, and televisions. They are also found in certain foods like fish, dairy, meat and eggs. PBDEs along with PCBs and dioxins are toxic, persistent, and bioaccumulative. These chemicals are also lipophilic, or “fat-loving,” which causes them to build up and store in fatty tissues and fluids like breast milk. Some consequences of high exposure to PBDEs include impaired development of the brain and thyroid, hearing deficits, delayed puberty, decreased sperm count, fetal malformations, and possibly cancer. Unlike most levels of contaminants, PBDE concentrations in breast milk are actually increasing.

FIGS. 12 and 13 show the concentrations of PBDEs around the world and in Swedish women, where the concentration is increasing. The United States has the highest levels of PBDE. The corresponding risk associated with the United States levels of PBDE is three times higher than the risk of the neighboring country Canada. Even the risk of countries with the lowest concentrations of PBDE is well above 100, which is completely unacceptable assuming a risk of 0 is safe. Risks are calculated with the United States' Environmental Protection Agency's Region 9. Preliminary Remediation Goals (PRG). The PRGs are risk-based tools which help in evaluating and cleaning up contaminant sites. Looking at the risks of countries like Japan and Germany we see that PBDEs in breast milk is a worldwide problem.

| | | Risk | |
|---|---|---|---|
| | | (out of 1 | |
| Country | PBDE (ppb) | million) | Source |
| Canada | 22 | 2912 | 1 |
| Finland | 2.3 | 304 | 2 |
| Germany | 6.6 | 874 | 1 |
| Holland | 3.3 | 437 | 1 |
| Japan | 1.4 | 185 | 1 |
| Norway | 2.8 | 371 | 1 |
| Sweden | 4 | 530 | 2 |
| UK | 8.9 | 1178 | 2 |
| USA | 73.9 | 9783 | 2 |

PCBs are found in industrial insulators and lubricants and have been banned since 1976. PCBs are known to cause cancer and nervous system problems. They have also caused infant death, birth defects, and brain damage. PCB exposure in the womb or during lactation is associated with decreased IQ, impaired psychomotor development, decreased immune function, and skin disease. It is a probable human carcinogen and concentrations in breast milk are even higher than PBDE concentrations (FIG. 14). Risks are calculated at thousands and even hundreds of thousands. The United States has a PCB concentration of 54 ppb which correlates to a risk of 7,148 and concentrations reach as high as 1,069 ppb in China, which is a risk of 141,512.

| | | Risk | |
|---|---|---|---|
| | | (out of 1 | |
| Country | PCB (ppb) | million) | Source |
| Austria | 381 | 50436 | 5 |
| Canada | 137 | 18136 | 5 |
| China | 1069 | 141512 | 5 |
| Finland | 189 | 25020 | 5 |
| Germany | 375 | 49642 | 5 |
| Hungary | 61 | 8075 | 5 |
| Netherlands | 253 | 33492 | 5 |
| Norway | 273 | 36139 | 5 |
| Russia | 197 | 26079 | 5 |
| Spain | 452 | 59835 | 5 |
| UK | 130 | 17209 | 5 |
| USA | 54 | 7148 | 11 |

Mercury is a metal found in soil, rock, air, and water, including drinking water. It is used in lamps, batteries, and other products and is released into air, water, and soil by some industries. Mercury is also used in many health industries, dental fillings (amalgam fillings), hospitals, laboratories, and pharmaceuticals as well as in glass and jewelry industries. A major source of mercury ingestion is from fish and seafood.

This metal is not lipid-soluble. It potentially harms brain development and function, slows growth, increases risk of learning problems, and causes mental retardation. Mercury can also cause developmental malformations and alters immune, reproductive, cardiovascular, and kidney function.

The Centers for Disease Control and Prevention recently reported data showing one out of every six women of childbearing age has mercury levels in their blood that the National Academy of Sciences considers unsafe for developing babies. One study in Japan on women who ate mercury contaminated fish in the 1950s found that some babies born to these women died within days of birth, while the mothers stayed healthy. They found mercury induced lesions on some areas of the adult brain while these same lesions were over the entire cortex of the baby's brain. This demonstrates the higher vulnerability of a developing infant to contaminants. The current average concentration of mercury in breast milk in Japan is 63 ppb, one of the highest in the world.

| | Mercury | |
|---|---|---|
| Country | (ppb) | Source |
| Austria | 35.8 | 8 |
| Brazil | 5.8 | 7 |
| Canada | 0.15 | 7 |
| China | 1.6 | 7 |
| Germany | 0.57 | 7 |
| Hungary | 1.4 | 7 |
| Italy | 13.94 | 9 |
| Japan | 63 | 7 |
| Philippines | 1.7 | 7 |
| Spain | 9.5 | 7 |
| Sweden | 3.3 | 7 |
| USA | 1.7 | 12 |

Lead is found in paint and was previously used in gasoline. Unlike most other contaminants, lead accumulates in mother's bones. It is drawn along with calcium into milk during lactation. It is more easily absorbed into growing bodies than fully developed bodies. Infants may absorb up to 50% of dietary lead, while adults may absorb only 10%, according to the literature (see references).

FIG. 5 shows the mean lead concentrations in breast milk around the world. Lead causes brain damage, affects a child's growth, damages kidneys, impairs hearing, and causes learning and behavioral problems. The US has an average concentration of 29 ppb lead in breast milk which carries a risk of almost 8,000. This risk is however dwarfed by Austria's risk of 40,548 and Italy's risk of 34,658.

| | | Risk | |
|---|---|---|---|
| | Lead | (out of 1 | |
| Country | (ppb) | million) | Source |
| Austria | 148 | 40548 | 7 |
| China | 8.7 | 2384 | 7 |
| Czech Republic | 1.7 | 466 | 7 |
| Egypt | 66 | 18082 | 7 |
| Germany | 15.5 | 4247 | 7 |
| Hungary | 14.9 | 4082 | 7 |
| India | 1.9 | 521 | 7 |
| Italy | 126.5 | 34658 | 7 |
| Philippines | 16 | 4384 | 7 |
| Spain | 0.11 | 30 | 7 |
| Sweden | 0.5 | 137 | 7 |
| USA | 29 | 7945 | 7 |

Dioxins can be found as a result of incineration and as chemical byproducts of some manufacturing processes. They are toxic to the developing endocrine system, cause birth defects in animals, disrupt reproductive development, and effect immune and hormone systems. The concentrations of dioxin found in breast milk may seem small compared to other chemicals since the concentrations are under 1 ppb, but dioxin is many times more toxic. FIG. 6 shows the various concentrations of dioxins around the world. In the U.S., a concentration of 0.0156 ppb of dioxin in breast milk yields a risk of 34,805. Countries all of the world have concentrations with risks in the tens of thousands range.

| | | Risk | |
|---|---|---|---|
| | Dioxin | (out of 1 | |
| Country | (ppb) | million) | Source |
| Austria | 0.014 | 31235 | 6 |
| Canada | 0.0211 | 47076 | 6 |
| China | 0.0027 | 6024 | 6 |
| Czech Republic | 0.0185 | 41275 | 6 |
| Finland | 0.0199 | 44399 | 6 |
| France | 0.0203 | 45291 | 6 |
| India | 0.0067 | 14948 | 6 |
| Italy | 0.0315 | 70280 | 6 |
| Japan | 0.018 | 40160 | 6 |
| Norway | 0.0128 | 28558 | 6 |
| Spain | 0.0255 | 56893 | 6 |
| Sweden | 0.018 | 40160 | 6 |
| UK | 0.021 | 46853 | 6 |
| USA | 0.0156 | 34805 | 6 |

DDT was used as a pesticide and enters the body through fruits, vegetables, fatty meat, fish, poultry, and contaminated drinking water. DDT degrades into DDE and DDD. DDT is known as a possible human carcinogen and may cause decreased fertility, kidney, and liver dysfunction, a weakening of the immune system, and various cancers. DDT contamination is prevalent world-wide (FIG. 17).

Even after DDT was banned in 1972, it remains at a concentration of 264.45 ppb giving a risk of 1,337 in the US. DDE is at an even higher concentration, 790.3 ppb. The reason DDE concentrations are still high after being banned for over 30 years is because of the prevalence of a high fat diet in the US. In France DDT concentrations go up to 2,283 ppb which is over 11,000 in estimated cancer risk. In developing countries, DDT use has increased in agriculture and malaria control. It is still used today in Africa, Asia, and Latin America for vector control.

| | | Risk | | | | |
|---|---|---|---|---|---|---|
| | | (out | | | | |
| | DDT | of 1 | | DDE | | |
| Country | (ppb) | million) | Source | (ppb) | Risk | Source |
| Canada | 20.15 | 102 | 4 | 644.5 | 3259 | 4 |
| Czech Republic | 1845 | 9330 | 3 | — | — | — |
| Finland | 110 | 556 | 4 | 567 | 2867 | 4 |
| France | 2283 | 11545 | 3 | — | — | — |
| Germany | 531 | 2685 | 3 | 906 | 4582 | 4 |
| Japan | 61.77 | 312 | 4 | 1600 | 8091 | 4 |
| Mexico | 1224 | 6190 | 4 | 4335 | 21923 | 4 |
| Norway | 338 | 1709 | 3 | — | — | — |
| Spain | 660 | 3338 | 3 | — | — | — |
| Sweden | 283 | 1431 | 3 | — | — | — |
| USA | 264.45 | 1337 | 4 | 790.3 | 3997 | 4 |

In addition to this list, an additional chemical has been found in breast milk. Perchlorate, a constituent of rocket fuel, is found in virtually all breast milk at concentrations five times higher than in cow's milk. It impairs the thyroid, slowing brain development, and cellular growth in a fetus or infant. This can result in lower IQs and neurological damage.

Some chemicals effect children directly like lead and mercury. Other chemicals start a chain of events that may result in cancers, cardiovascular disease, or diabetes later in life. This vulnerability may even be passed down to future generations and can cause gene mutations permanently engrained in DNA from generation to generation. Contaminants in breast milk are a lasting problem that will plague mother's worldwide for generations to come. Yet breast milk provides essential nutrients needed for a full and healthy life. Our only option is to continue feeding this poisoned potion to our infants and try to reduce the amount of contaminants to the best of our abilities.

[image]

[image]

[image]

[image]

[image]

[image]

[image]

### SUMMARY OF THE DISCLOSURE

Briefly stated, with a carbon-based filtration system, binding and removal of contaminants and other chemicals is accomplished whereby breast milk may be substantially improved in terms of noxious chemicals and toxic elements reducing the likelihood of transferring body burdens in infants.

According to a feature of the present disclosure a method for mitigating deleterious impacts of contaminants in breast milk, which comprises, in combination: providing a carbon-based filtration system between a breast milk source and a recipient of the milk; processing the milk to bind and remove contaminants and chemical agents having potentially negative impacts upon the recipient; by, passing the milk through a carbon-based filtration system including anionic and cationic resins; and finishing by at least one of making the filtered milk available to a recipient and storing it in a receptacle.

Moreover, the present disclosure discloses a filtration system, comprising, in combination: a filtration medium housed in a cartridge; whereby the filtration medium is in fluid communication with a source of milk and a receptacle for housing filtered milk, and whereby toxins are removed from milk contacting the filtration medium by at least one of binding, mechanical, and ionic separation; and whereby the cartridge is modular.

Finally, a novel enhanced toxic insult mitigation protocol is disclosed, which comprises, in combination: screening pregnant patients; measuring toxin levels; comparing the toxin level to established values; evaluating projected body burdens; establishing ameliorative measures; and advising patients on execution of the same.

### BRIEF DESCRIPTION OF THE DRAWINGS

The above-mentioned features and objects of the present disclosure will become more apparent with reference to the following description taken in conjunction with the accompanying drawings wherein like reference numerals denote like elements and in which:

FIG. 1 is a perspective view of a nipple shield filter device showing a position of the filter in the nipple shield filter.

FIG. 2 is a perspective view of a nipple shield filter device showing an alternative positioning of the filter within the nipple filter device.

FIG. 3 is a cross-sectional view of a nipple shield filter device showing one configuration of the filter within the nipple filter device.

FIG. 4 is a perspective view of a nipple shield filter device showing a plurality of filters positioned in the nipple shield filter device.

FIG. 5 is an exploded perspective view of a filtering baby bottle.

FIG. 6 is a perspective view of a nipple filter device showing an alternate positioning of the filter in the nipple filter device.

FIG. 7 is a perspective view of a nipple filter device showing an alternate configuration of the filter in the nipple filter device.

FIG. 8 is a sectional schematic view of a breast milk pump showing a positioning of the breast milk pump filter within the breast milk pump.

FIG. 9 is a sectional view of an embodiment of a “breast pump filter cartridge holder.”

FIG. 10 is an exploded view of an embodiment of a “breast pump filter cartridge holder.”

FIG. 11 is a chart showing Human Breast Milk is Contaminated, from the Environmental Research Foundation, August 1990.

FIG. 12 is a chart showing PBDEs Breast Milk and Fat Samples Around The World, from Healthy Milk, Healthy Baby, NRDC, March 2005.

FIG. 13 is a chart showing dramatic increase in levels of fire retardants in Swedish women's bodies, 1972-1997.

FIG. 14 is a chart showing Concentrations of Seven Indicator PCBs in Breast Milk Around the World (1990s), from Healthy Milk, Healthy Baby, NRDC, March 2005.

FIG. 15 is a chart showing Mean Lead Concentration in Breast Milk by Location, Healthy Milk, Healthy Baby, NRDC, March 2005.

FIG. 16 is a chart showing Dioxins and Furans in Breast Milk Around the World (Later 1980s and 1990s), Healthy Milk, Healthy Baby, NRDC, March 2005.

FIG. 17 is a chart of DDT and DDE in Breast Milk and Adipose Tissue Around the World (1990s-2000s), Healthy Milk, Healthy Baby, NRDC, March 2005.

### DETAILED DESCRIPTION

The present inventor has discovered ways to safeguard developing infants from a newly documented body burden—namely the breast milk of their mothers, as discussed above. Although mitigation and extenuation of toxicity is accomplished by the instant disclosure, there is no adverse impact either on nutrition or mother-child harmony.

Significantly, it has been discovered that novel breast pump filtration systems, methods, and processes can do this without interrupting positive aspects of the breast-feeding protocol that is advocated by many neo-natal specialists. By providing options to pump and store, pump and feed, filter while feeding, and pump and filter, the instant teachings do not interfere with, but rather enhance, the breast-feeding process.

In contrast with the filtration described in published U.S. Patent Application 2004/0178162A to Zucker-Franklin, entitled “Devices and Methods for Removal of Leukocytes from Breast Milk,” incorporated herein by reference, the present approach is directed to the removal of dissolved or suspended organic compositions or inorganic ions rather than size filtration to remove bacteria. The filtration media useful for the removal of leukocytes will not be effective for the removal of organic and inorganic toxins as described herein. However, different filtration media can be combined such that organic and/or inorganic toxins are removed as well as leukocytes.

In general, any breast shield design can be adapted for incorporation of a suitable filtration medium. However, it may be desirable to adjust the shape of the device to better provide for placement of the filtration medium without interfering with the placement of the device on the nursing mother. Similarly, the placement of the filtration medium can be selected to provide proper fit of the device.

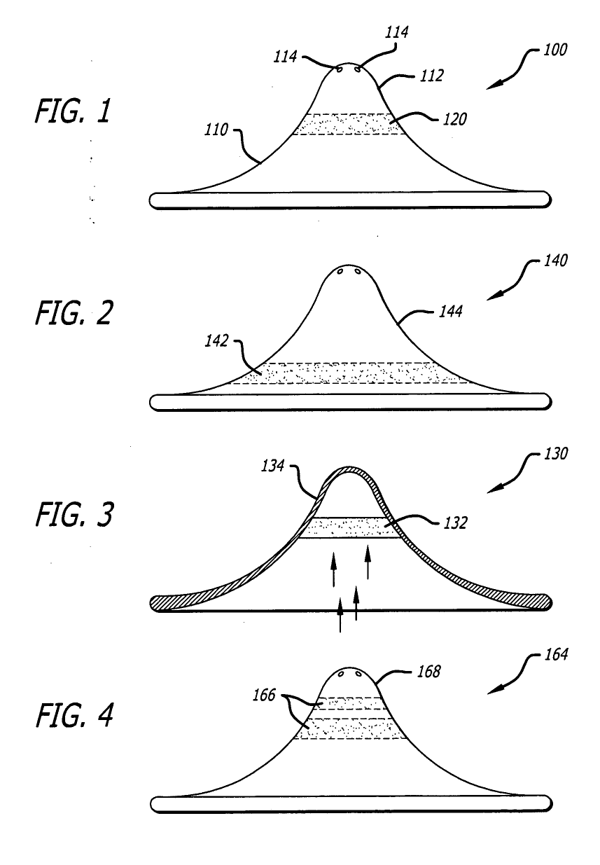

Referring to a representative embodiment in FIG. 1, a filtering nipple shield 100 comprises a nipple structure 110 with a tip 112. The tip has one or more holes 114 permitting passage of breast milk such that an infant may intake the breast milk. The filtering nipple shield 100 also comprises filter media 120 interior to the nipple shield 100 such that breast milk flows through the filter media 120 on its way to the holes 114. Nipple structure 110 can be shaped to conform to a mammalian female areola and nipple. As the mammalian breast can vary in shape and size, nipple shield 100 can take a variety of forms to accommodate these variations in breast size and shape. Filtering nipple shield 100, when placed over the areola and nipple of a mammalian breast, is shaped such that when it is sucked on by an infant, suction is created between the nipple shield 100 and surface of the mammalian breast. Commercial nipple shields are commercially available from companies such as Medela Inc. and Ameda. Commercial nipple shields can be adapted as nipple structures 110.

Filtering nipple shield 100 is generally made of flexible material such that nipple structure 110 can conform somewhat to the mammalian breast shape and size. Suitable materials for the nipple structure 110 can include, for example, rubber, latex, silicon, or the like, or combinations thereof.

Referring to FIG. 2, filtering nipple shield 140 comprises a filtering medium 142 at an alternative location in relationship with nipple structure 144. The filter media within the nipple shield can comprise a material that is capable of filtering-out endocrine disruptors such as polybrominated diphenyl ethers, polychlorinated biphenyls, dioxins, dibenzofurans, perchlorates, phthalates, and/or heavy metals and radionuclides. Suitable filtration media for the removal of organic compounds include, for example, activated carbon.

The activated carbon can be within a porous block material with a polymer binder, such as described in U.S. Pat. No. 4,753,728 to VanderBilt, et al., entitled “Water Filter,” incorporated herein by reference. However, the pressure drop across such a block structure can lead to undesirable nursing difficulties. Thus, it may be more desirable to place a granular activated carbon material within a porous structure that prevents the migration of the activated carbon while providing flow through the porous structure. Food grace activated carbon suitable for these applications is sold commercially by Calgon (Filtrasorb®) and U.S. Filter (AquaCarb® and BevCarb®).

Active carbon filters can be effective in removing organic contaminants and endocrine disruptors such as halogenated hydrocarbons including PCB's and PBDE's, dioxins, dibenzofurans, and perchlorates, phthalates, and some heavy metals such as arsenic complexes, chromium complexes, and mercury complexes. The activated carbon filter material can be hydrophobic or hydrophilic, and can be granular with a mesh size selected to avoid migration of the activated carbon while providing a suitable surface area to remove desired contaminants.

The addition of cationic and anionic resins that absorb cations and anions assists in filtering radionuclides and heavy metals from the breast milk. For example, radium can be removed by including sorbents, for example, acrylic fibers or resins impregnated with manganese dioxide, and non-sodium cation exchangers such as hydrogen ions and calcium ions. Carbion™ ion exchanger, available from Lenntech, for example, can be used as an ion exchanger to remove heavy metals.

The filter media can be contained within a porous membrane that allows for relatively easy flow of breast milk through the filter material and filter media. The filter material can be used to keep the filter media localized and contained in a disc or packet, or held within a porous silicon or porous rubber structure. Suitable materials for the filter material include, for example, a woven material, such as polyester or other woven polymer or a nonwoven material, such as a porous plastic material.

The porosity is chosen to keep the granular filtration medium within the membrane while providing for suitable milk flow. The membrane with the filtration medium can be molded into the nipple shield, attached within the nipple shield through welding, adhesive bonding or the like, or wedged releasably within the nipple shield with friction. The nipple shield can be discarded after each use, or cleaned and/or sterilized for reuse.

Referring to FIG. 3, filtering nipple shield 130 comprises filtering media 132, in this embodiment, positioned in the tip 134 of the filtering nipple shield 130. The filtering nipple shield 130 is shown in a cross-sectional view such that the directional flow of breast milk through the filter media 132 is shown by the positioning of the arrows. FIG. 4 demonstrates another embodiment of a filtering nipple shield 164, wherein a plurality of filtering media 166 is placed within the filtering nipple shield tip 168. The plurality of filtering media 166 is placed sequentially within the nipple tip 168, such that the breast milk passes through a plurality of filters prior to being ingested by a feeding infant.

In general, the filter element can be permanently or releasably connected to the remaining portions of the nipple shield. Permanent connections can be formed with molding or adhesives or the like. Releasable connections can be formed with friction elements such that the filter remains in position during use but can be pulled out when desired. Thus, if the filter has a significantly longer or shorter lifetime than the other portions of the nipple shield, the elements can be independently replaced if the filter element is releasable attached.

Filtering nipple shield embodiments, such as these described above, provide for direct filtering of breast milk as the milk is ingested by a suckling infant. Alternatively, the milk can be collected for subsequent ingestion by an infant. In these embodiments, the breast milk can be filtered during the collection process or at the point of ingestion.

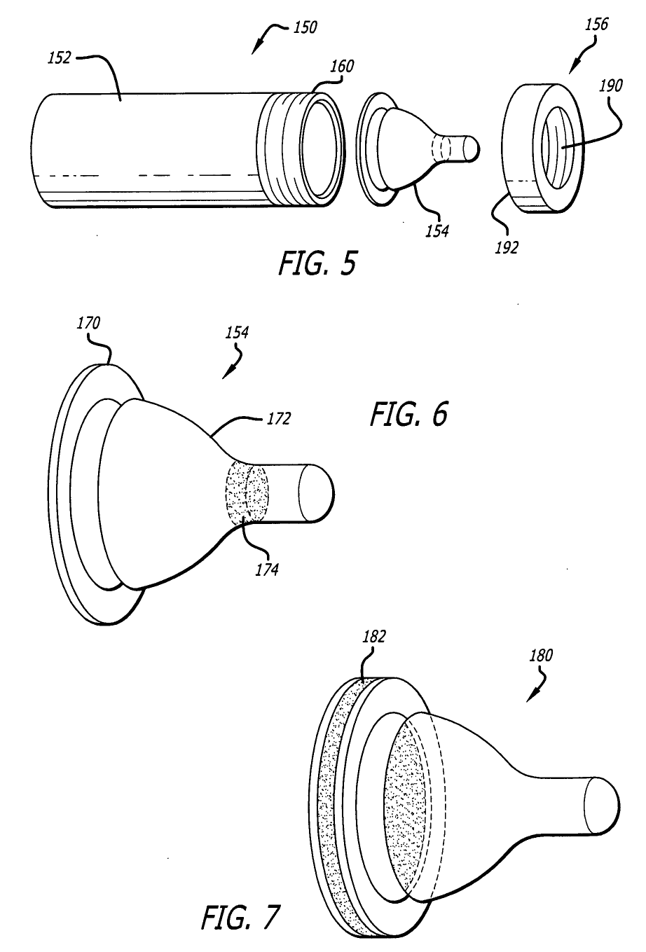

For example, a filter can be attached to a bottle that holds that breast milk for ingestion. These filtering bottles similarly can be used to filter other liquids, such as cow's milk, sheep's milk, juices, or the like prior to ingestion. In general, the filter medium can be placed along the flow path from the storage portion of the bottle to the bottle tip from which the liquid is consumed. A representative embodiment is presented in FIG. 5.

Referring to FIG. 5, filtering bottle 150 comprises a storage compartment 152, bottle nipple 154 and cap 156. Breast milk that has been pumped and saved for future use or another liquid can be poured into filtering bottle 150 after sterilization. Storage compartment 152 can have conventional dimensions for easy holding and for storage of an appropriate quantity of liquid. Storage compartment 152 can comprise a disposable bag or the like to hold the liquid rather than directly placing the liquid into the storage compartment. Storage compartment 152 has an attachment portion 160 for the attachment of cap 156. Attachment portion 160 can comprise threads or the like for the attachment of embodiments in which cap 156 comprises mated threads. Alternatively, a clamp or the like can be used to secure cap 156 with attachment portion 160 in which cap 156 and attachment portion 160 have suitable flanges to engage the clamp. Similarly, any other suitable attachment structure can be used. FIGS. 6 and 7 show alternate bottle nipples 154, 180 that can be used with storage compartment 152.

Referring to FIG. 6, bottle nipple 154 comprises lip section 170, nipple portion 172 extending from lip section 170 and filter portion 174 within nipple portion 172. Lip section 170 has suitable dimensions for interfacing with attachment portion 160 and the positioning between attachment portion 160 and cap 156 such that bottle nipple can be held in place. Filter portion 174 can have similar structure and filter compositions as filtering medium 142 in the breast shield, as described above.

However, the placement of the filter medium can be positioned without regard for interference with the placement of the nipple portion over the nursing mother's breast since bottle nipple 154 is just placed on a bottle. Thus, referring to FIG. 7, bottle nipple 180 comprises filter element 182 placed across the mouth of bottle nipple 180, as an alternative or in addition to the placement of the filter element further toward the tip of the bottle nipple. As with the filtering nipple shield, bottle nipple 180 can comprise a plurality of filter elements 182. Also, filter element 182 can be secured across the mouth of the storage compartment without direct attachment with bottle nipple 154.

Referring again to FIG. 5, cap 156 comprises orifice 190 and cap attachment section 192. Bottle nipple 154, 180 fits through orifice 190 for attachment to storage compartment 152. Cap attachment section 192 can comprise threads mated for engaging attachment portion 160 or other suitable structure for engaging attachment portion 160 directly or with a clamp or the like. In alternative embodiments, the filtering baby bottle may not include a cap element. For example, the lip section of the nipple portion can have an elastic seal that extends over and releasably grips the attachment portion of the storage compartment.

For use, storage compartment 152 and cap 156 are attached such that lip 170 or a separate gasket or the like provides a seal so that liquid does not leak out of bottle 150. When the infant sucks on bottle nipple 154 to obtain milk from filtering bottle 150, the milk passes through filtering portion 174. Upon emptying the bottle, bottle nipple 154 and/or filter portion 174 can be removed and discarded.

Alternatively, bottle nipple 154 and/or filter portion 174 can be cleaned, sterilized, and reused. In further embodiments, new or sterilized filter portion 174 can be placed in the interior of bottle nipple 154. Storage compartments 152 can be formed of suitable plastics. Bottle nipple 154 and filter portion 174 can generally be made of similar corresponding materials described above with respect to the nipple shield.

In a further embodiment, a filter is placed as an integral part of a breast milk pumping device. The breast milk pumping device generally can comprise any type of filtration medium to filter the breast milk. In some embodiments, the filtration medium in the breast milk pump comprises activated carbon and/or an ion absorptive medium, such as an ion exchange resin. The activated carbon filter material can be granular with a mesh size in the range from 0.025 mm to 4.75 mm in width. The filter media 40 can be contained in a filter packet, where the covering filter material allows for passage of the filtered breast milk. The packet material can be comprised of nonwoven and/or woven material. Breast milk pumps are available from manufacturers, such as Medela Inc. and Ameda. Commercial designs can be adapted for filtration or new designs can be used.

In general, a filtering breast milk pump comprises a collection reservoir, a collection cup, a filter in the flow path from the collection cup to the collection reservoir and a pump. The collection reservoir can be any suitable size and shape. The collection cup generally is designed to fit reasonably and comfortably over a female mammalian breast for collecting the milk. The cup generally has a neck extending from the cup that leads to a channel directed to the reservoir. The filter is positioned within the flow path from the woman's breast to the reservoir. Thus, the filter can be placed, for example, in the neck of the cup or in the channel leading to the reservoir.

The pump can be connected to the remaining portions of the device in a range of configurations. Many configurations have been described. The pump can be a manual pump in which the user pumps the device to provide the desired degree of pressure differential. Manual pumps generally can have a handle connected to a baffle, an elastic bladder or the like to perform the pumping action. Alternatively or additionally, a motorized pump can be used. A motorized pump has the advantage that a person does not have to provide the pumping action.

An example of a breast pump construction that can be adapted for manual or automatic suction pumps is described further in U.S. Pat. No. 4,759,747 to Aida, et al., entitled “Breast Pump Including Pressure Adjusting Means,” incorporated herein by reference. Another representative breast pump design is discussed in U.S. Pat. No. 6,110,141 to Nuesch, entitled “Breast Pump Overflow Protection for an Apparatus for Sucking a Body Fluid Off,” incorporated herein by reference. The present filtration designs for filtering milk prior to entering the reservoir are in stark contrast with designs intended to prevent fouling of the pump, which generally are designed to prevent passage of milk rather than filtering the milk.

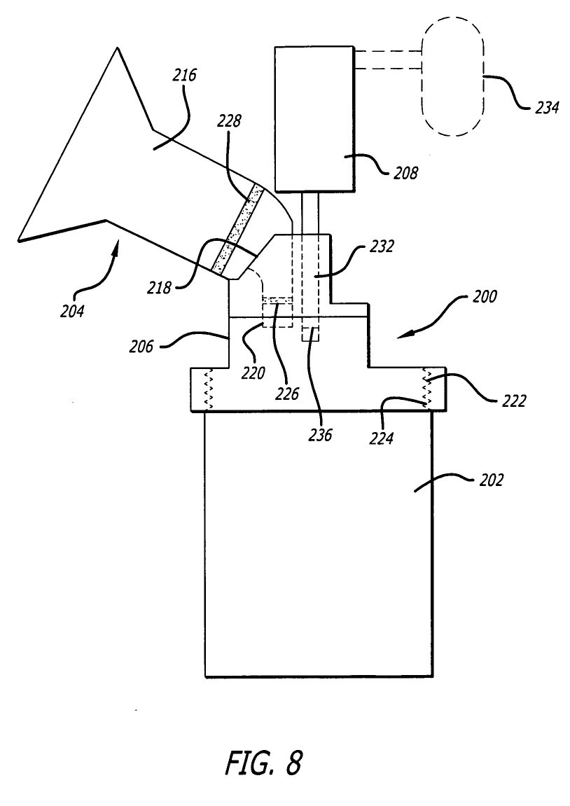

A schematic view of a representative embodiment of a filtering breast milk pump is shown in FIG. 8. Breast pump 200 comprises reservoir 202, funnel shaped cup 204, manifold 206, and pump 208. Reservoir 202 holds the filtered breast milk. The reservoir can be accessed for the removal of the filtered breast milk and for subsequent cleaning of the reservoir, if desired. Cup 204 is designed to fit over the breast of the nursing mother. Cup 204 has funnel shaped cone 216 that tapes into neck 218. Neck 218 transitions into conduit 220 leading to reservoir 202 or similarly is fluidly connected to such a conduit. Milk flowing through the neck is collected in the reservoir.

Manifold 206 provides for connections between reservoir 202, cup 204, and pump 208. Manifold 206 can have connection 222 such as screw elements for the removal reservoir 202 from manifold 206, in which case reservoir 202 comprises mated screw threads 224. It can be advantageous to provide a screw lid to close the reservoir to obviate the need to transfer the milk to a separate storage container. In other embodiments, manifold 206 can be fixed to a reservoir with a resealable opening to provide access for the removal of the filtered milk.

A filter element generally is located within the flow from cup 204 to reservoir 202. As shown in FIG. 8, filter element 228 is shown in cup 204, and filter element 226 is shown in conduit 220. Pump 200 can include one or both representative filter elements 228, 226 or other filter elements along the flow pathway. Generally filter elements 228, 226 can be removed for cleaning and/or replacement. Filter elements 228, 226 can be formed from similar materials and similar filtration media as filter elements described with respect to FIGS. 1-7.

Pump 208 is fluidly connected to pump conduit 232 that provided for creating negative pressure within reservoir 202. Pump 208 can be a motorized pump or a manual pump. An optional manual squeeze ball 234 is shown in phantom lines in FIG. 8. An optional air filter 236 is shown within pump conduit 232 to keep milk from entering the pump. Pumping is performed as needed, and the filter elements generally do not significantly alter the pumping process.

Generally, the devices described herein as well as other potential devices can be used to practice a method of removing organic toxins and/or inorganic toxins, such as halogenated endocrine disruptors, phthalates, radionuclides, heavy metals, and other toxins from breast milk.

As discussed above, the method can be used for the direct filtration during the suckling process of an infant or for the filtration of stored milk at collection, at delivery or during some subsequent period between collection and delivery. In some embodiments, the filter comprises a filtration medium with activated carbon, since activated carbon is effective at the removal of halogenated organic compounds. However, other suitable filtration media can be used.

In one embodiment, filtering nipple shield 100 can be placed over an areola and nipple region of a female mammalian breast. The infant would suck on the nipple thereby creating suction between the nipple shield and the mammalian breast such that enough suction is created to cause milk to flow from the mammalian nipple through the filtering nipple shield 100. The breast milk flows through the filter media prior to exiting the filtering nipple shield 100.

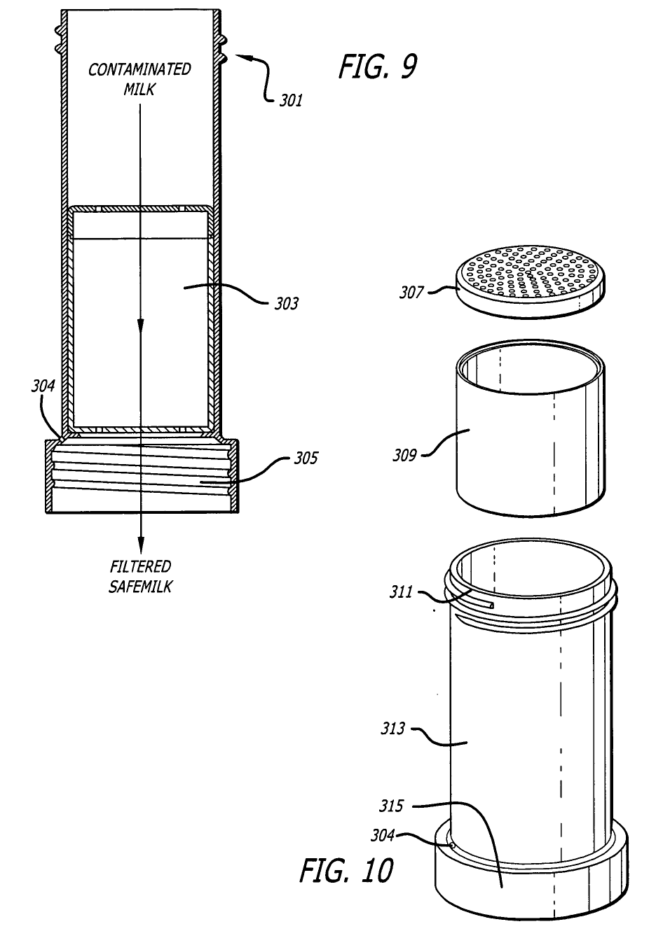

Turning now to FIG. 9, a schematic of a breast pump filter cartridge holder 300 is offered for consideration, in exemplary fashion, as opposed to limiting the teachings of the present disclosure. In an embodiment, for example, according to functional prototypes of the instant teachings (available from SAFEMILK, LLC of Venice, CA 90291), male fitting 301 attaches to a pump (such as shown in FIG. 8, and otherwise developed, available, or improved) which removes contaminated milk from a breast (not shown) into filter cartridge 303 filled with activated carbons, and anionic and cationic resins, which bind to and thereby remove contaminants, as described and charted and entabulated above.

Female fitting 305, and interchangeable variations as would be known to those of skill in the art fittingly engages both known (see above) and later developed bottles for infants or those needing to drink breast milk, in addition to other receptacles, bags, cold storage media and the like mechanisms for maintaining post-filtered product in a state where it may be consumed. Back pressure orifice 304 prevents buildup of pressure between breast pump filter cartridge holder 300 and the bottle by providing an avenue for displaced air to escape as milk flows from the breast pump filter cartridge holder into the bottle.

This is neither limited to post-filtered product, nor milk to be consumed right away. For example, using known technology (or that which is proprietary but later becomes available) one can ‘freeze-dry’, evaporate and bubble, or foam and dehydrately store aliquots of filtered milk or milk to be filtered. See for example, U.S. Pat. No. 6,691,771—which is incorporated expressly by reference herein as if fully set forth. According to the '771 patent, foamed glass and compositions obtained thereby are explained, which could be effectively used with the teachings of the present disclosure.

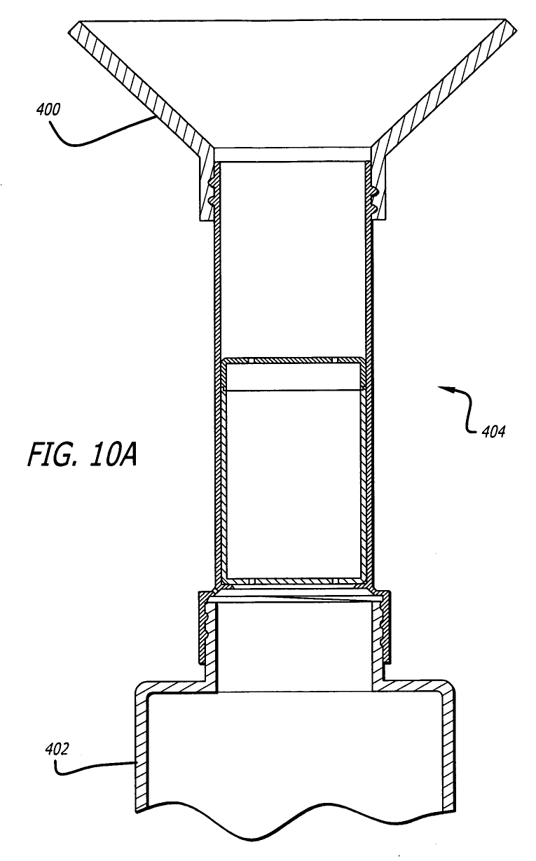

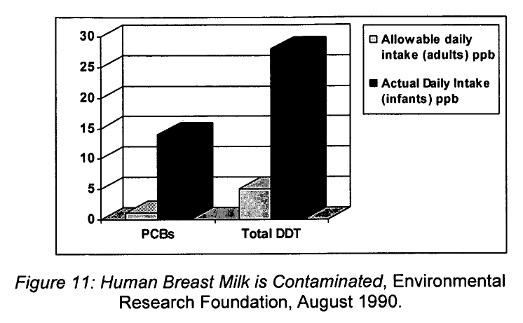

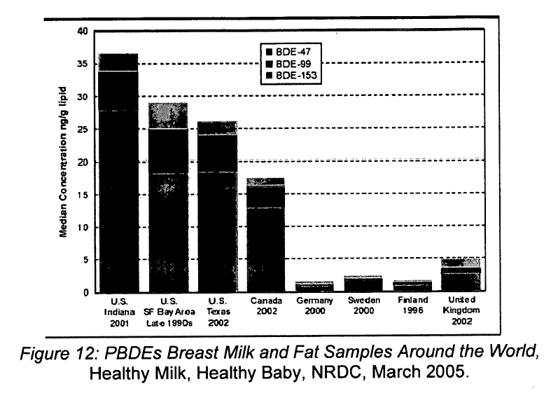

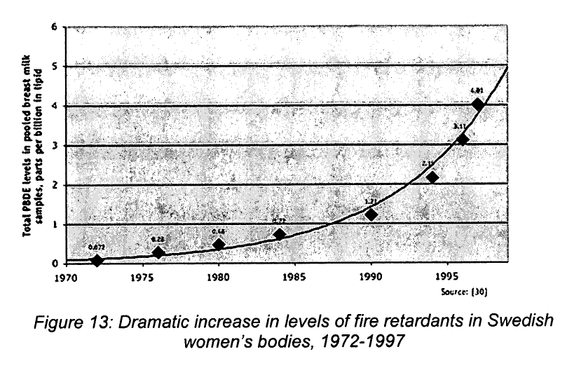

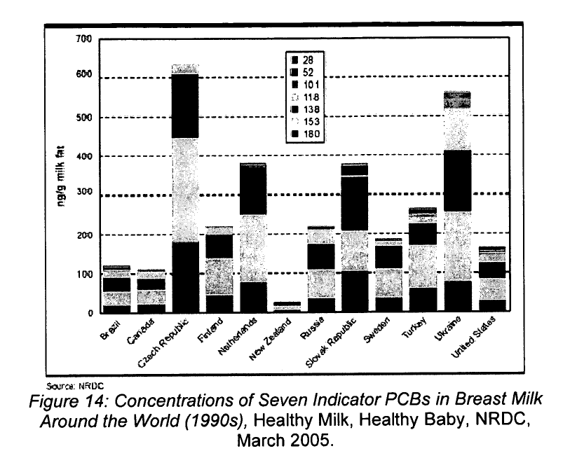

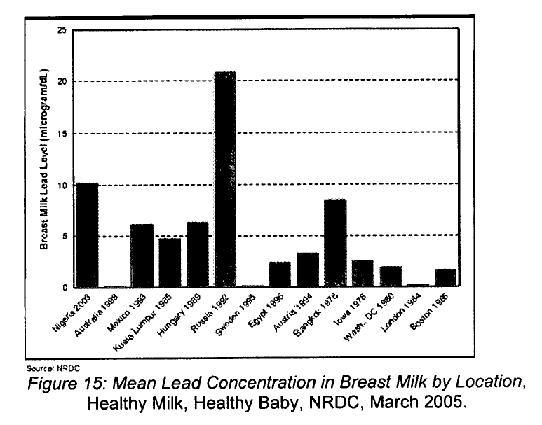

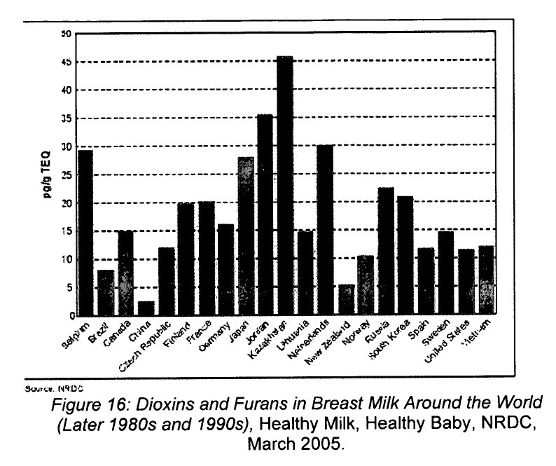

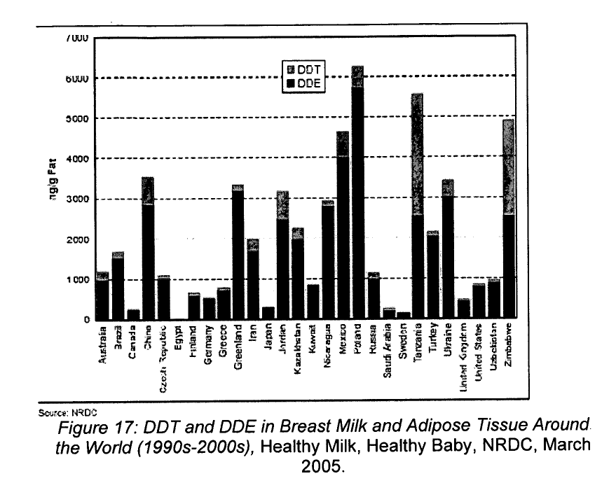

FIG. 10 likewise schematizes a breast pump filter cartridge holder, in exploded view, further comprising cap mechanism 307, which allows milk to pass though to filter cartridge 309, male fitting 311 which attaches to pump, as discussed above and female fitting attaching to bottle, collection reservoir or any other desired container.

FIG. 10A similarly demonstrates the utility of the present disclosure in previously recovered breast milk. Milk funnel 400 is connected to breast pump filter cartridge holder 300, which is connected to an appropriate storage compartment 402. Milk placed into milk funnel 400 flows into and is filtered in breast pump filter cartridge holder 300. Either a pump, the force of gravity, or both causes the milk to flow from milk funnel 400 into and through breast pump filter cartridge holder 300. From there, it flows into an appropriate storage compartment 402 as previously described.

Those skilled in the art understand that these mechanisms may be readily interchanged with others, which perform the same function in the same way to achieve the same result, and that the chemicals and contaminants to be removed may likewise be expanded or contracted as more data becomes available.

While the apparatus and method have been described in terms of what are presently considered to be the most practical and preferred embodiments, it is to be understood that the disclosure need not be limited to the disclosed embodiments. It is intended to cover various modifications and similar arrangements included within the spirit and scope of the claims, the scope of which should be accorded the broadest interpretation so as to encompass all such modifications and similar structures. The present disclosure includes any and all embodiments of the following claims.

1. A method for mitigating deleterious impacts of contaminants in breast milk, which comprises, in combination:
providing a carbon-based filtration system with cationic and anionic resins between a breast milk source and a recipient of the milk;
processing the milk to bind and remove contaminants and chemical agents having potentially negative impacts upon the recipient; by,
passing the milk through a carbon-based filtration system including anionic and cationic resins; and
finishing by at least one of making the filtered milk available to a recipient and storing it in a receptacle.

2. The method of claim 1, wherein the providing step further comprises:
screening the breast milk source for at least one of contaminants and undesired chemical agents.

3. The method of claim 1, the processing step further comprising utilizing a breast pump detachably engaged with the carbon-based filtration system.

4. The method of claim 1, the processing step further comprising utilizing a readily detachable funnel-shaped member matingly engaged to the carbon-based filtration system, thereby enabling flow of milk driven in part by gravity.

5. The method of claim 1, further comprising emplacing at least one of a nipple-shield, a breast pump-filter and a means for extracting and passing through milk at the breast milk source.

6. The method of claim 1, further comprising measuring contaminants and extracted chemical entities.

7. The method of claim 1, further comprising at least one of a filtering nipple shield, a filtering bottle, a filtering breast pump and alternate means for filtering.

8. The method of claim 6, further comprising planning a regimen to manage the body burden of the recipient to minimize insult and injury.

9. A filtration system, comprising, in combination:
a filtration medium housed in a cartridge;
whereby the filtration medium is in fluid communication with a source of milk and a receptacle for housing filtered milk; and
whereby toxins are removed from milk contacting the filtration medium by at least one of binding, mechanical and ionic separation; and
whereby the cartridge is modular.

10. The filtration system of claim 9, further comprising a plurality of male/female coupling attachments, whereby the cartridge is readily detachable.

11. The filtration system of claim 10, further comprising activated carbon, cationic and anionic resins.

12. The filtration system of claim 11, wherein the modular cartridge matingly engages a breast pump and a receptacle.

13. The filtration system of claim 11, where the modular cartridge is disposable.

14. The filtration system of claim 11, wherein the modular cartridge matingly engages a readily detachable funnel-shaped member and a receptacle, thereby enabling flow of milk driven in part by gravity.

15. A novel enhanced toxic insult mitigation protocol, which comprises, in combination:
screening pregnant patients;
measuring toxin levels;
comparing the toxin level to established values;
evaluating projected body burdens;
establishing ameliorative measures; and
advising patients on execution of the same.

16. The protocol of claim 15, wherein the screening step is done after giving birth.

17. The protocol of claim 16, further comprising designing a breast milk filtration regimen.

18. The protocol of claim 17, further comprising storing patient data on a computer-interfaceable medium.

19. The protocol of claim 18, wherein patient data is accessible over the Internet.

20. The protocol of claim 19, further comprising a wireless interface.

21. The protocol of claim 15, further comprising providing a carbon-based filtration system between a breast milk source and a recipient of milk.

22. The protocol of claim 21, the carbon-based filtration system further comprising anionic and cationic resins and at least one of a filtering nipple shield, a filtering bottle, a filtering breast pump and an alternate means for filtering.
